(19) United States
(12) Patent Application Publication
(10) Pub. No.: US 2020/0197450 A1
(43) Pub. Date: Jun. 25, 2020

(54) One kind of Live Bacteria Preparation of Bacillus Coagulans and Preparation Method thereof

(71) Applicant: Zhifeng Han, Jiamusi (CN)

(72) Inventor: Zhifeng Han, Jiamusi (CN)

(21) Appl. No.: 16/804,022

(22) Filed: Feb. 28, 2020

(51) Int. Cl. A61K 35/742; A61K 9/20; A61K 9/28

(52) U.S. Cl. CPC A61K 35/742; A61K 9/2873; A61K 9/2059; A61K 9/2095

(57) ABSTRACT

One kind of live bacteria preparation of bacillus coagulans, the aforesaid live bacteria preparation is in the form of tablets; such live bacteria tablet is made from the bacteria powder of bacillus coagulans HQ-1, subsequently coated with gelatin and cooled, then made with other pharmaceutical auxiliary ingredients. The preparation method of such live bacteria preparation includes the following steps: preparing the pellet core coated with the bacteria powder; preparing the gelatin solution; wrapping the gelatin solution outside the pellet core, cooling to obtain the hard capsule; preparing the powder or pill with other pharmaceutical auxiliary ingredients and syrup; pressing to form the tablets. Such live bacteria tablet can fully protect the effective ingredient bacteria powder in the stomach and intestine, allowing the coat to be melt when reaching the affected area, so as to ensure a high utilization rate of bacteria powder.

### TECHNOLOGY DISCIPLINE

This invention affiliates to the technology discipline of biomedicine, in particular, a kind of Live Bacteria Preparation of Bacillus Coagulans and Preparation Method thereof.

### TECHNICAL BACKGROUND

Given the range of available technologies, Bacillus coagulans tablet (such as the ‘Bacillus Coagulans producing Plasmin and the Preparation Method of Plasmin and Live Bacteria Tablet’ published m Chinese Patent No. (CN109706092a) has been widely used for auxiliary treatment of digestive tract diseases, such as pylorus, duodenum, jejunum, etc. In the course of use, it has been noticed that the formulation of live bacteria tablet which is in line with medicative effects, regardless of the specific formula, invariably fail to could not be accurately used in the affected areas of pylorus, duodenum or jejunum, due to the difficulty in grasping live bacteria tablet's gastric acid tolerance, resulting in a lower actual effect than the expected counterpart.

### CONTENT OF INVENTION

In order to solve the aforesaid technical challenges, this invention provides a new kind of live bacteria preparation of bacillus coagulans and preparation method thereof, specified as below:

One kind of live bacteria preparation of bacillus coagulans, the aforesaid live bacteria preparation is in the form of tablets; such live bacteria tablet is made from the bacteria powder of bacillus coagulans HQ-1, subsequently coated with gelatin and cooled, then made with other pharmaceutical auxiliary ingredients.

The preparation method of such live bacteria preparation includes the following steps:

1) Take sucrose and purified water for heating and dissolving, and then cool down to obtain the syrup, ready for follow-up use;

2) Under room temperature, put the starch in the granulation, and power on the granulator; Meanwhile, spray syrup and the bacterial powder of bacillus coagulans HQ-1 until the required pellet cores are produced, and then take them out;

3) The core of the pellets shall be cooled under a temperature of 5-10° C. and be screened for follow-up use;

4) Take the medicine solid gelatin in the warm water bath and then cool it to room temperature to obtain the gel, ready for follow-up use;

5) The gel shall he heated to obtain the solution;

6) Take the core of the pellet produced in the step 3, power on the pelletizer. Meanwhile, spray the solution made in the Step 5. Whenever the mixture is even, spray the cold gas into the granulator till the gel is solidified, and obtain the pellet core wrapped with a hard capsule shell;

7) Weigh and take the pharmaceutical auxiliary ingredients and pellet core in line with the formula proportion, spray the syrup made in step 1) during the granulating process. Whenever the mixture is even, either pills or powder will be obtained in line with the size of pills.

In step 2), the starch, the individual mass ratio of sucrose and bacterial powder in the pellet core is subject to the tablet formula.

In step 7), the individual mass ratio of the pharmaceutical auxiliary ingredients, sucrose, starch and bacterial powder in the tablet is subject to the tablet, formula.

The idea of this invention is that, based on the tablet formula, change only the physical form of the bacterial powder in the tablet, enabling the bacterial powder to be dissolved in the area of intestinal canal under the protection of the hard capsule shell. In the course of production, each components is subject to the formula, and be put in various process stages.

Under room temperature of 25° C., the core effective components of the tablet is obtained by wrapping the bacterial powder with starch pill core and syrup.

Subsequently, the gelatin used for preparing the capsule is prepared into a solution. During this process, the preparation method of medicinal hard shell capsules is usually made by swelling the solid gelatin in a 50° C. water bath and then cooled to room temperature to obtain the gel. The solution is obtained by heating. Due to its properties, the temperature is ought to be between 35 C˜40 ° C. , it will gelatinize in case of less than 35° C. and dissolve in case of more than 40° C.

Spray the solution on the surface of the pellet core until it is even. Then spray cold air (e.g. carbon dioxide gas at about 10° C.), the pellet core covered with hard shell capsule shall be obtained.

Out of the aforesaid production steps, preparation of the pellet core needs to control the he particle size, followed by a screening step, so as to obtain the pellet core of small particles.

In the course of tablet pressing, according to the quality and size of tablet, a certain number of pellets shall be chosen and wrapped by pharmaceutical excipients.

In the powder, pill or tablet prepared by this method, starch and sucrose are common excipients in the food/drug preparation process. In major cases, they have basically no negative impact on the quality of gastrointestinal diseases.

In terms of the structure and preparation method, this live bacteria tablet has adopted a brand-new design. Given the existing equipment, it is suitable for the formulation of multi-purpose live tablet. Such live bacteria tablet can filly protect the effective ingredient bacteria powder in the stomach and intestine, allowing the coat to be melt when reaching the affected area, so as to ensure a high utilization rate of bacteria powder.

### SPECIFIC IMPLEMENTATION STEPS

This case is further specified in combination with the embodiment as follows: In this case, the live bacteria preparation of bacillus coagulans is a tablet; its formula can be the counterpart of live bacteria tablet, in accordance with the existing multi-genre active ingredients.

The live bacteria tablet is made from the bacterial powder of bacillus coagulans HQ-1, coated with gelatin and cooled, and then made with other pharmaceutical auxiliary ingredients.

The preparation method of the live bacteria preparation includes the following steps:

1) Take sucrose and purified water for heating and dissolving, and then cool down to obtain the syrup, ready for follow-up use;

2) Under room temperature, put the starch in the granulation, and power on the granulator; Meanwhile, spray syrup and the bacterial powder of bacillus coagulans HQ-1 until the required pellet cores, are produced, and then take them out;

3) The core of the pellets shall be cooled under a temperature of 5-10° C. and be screened for follow-up use;

4) Take the medicine solid gelatin in the warm water (50° C.) bath for two hours (mass concentration is bigger than 2%) and then cool it to room temperature to obtain the gel, ready for, follow-up use;

5) The gel shall be heated under the temperature of 40° C. to obtain the solution;

6) Take the core of the pellet produced in the step 3, power on the pelletizer. Meanwhile, pray the solution made in the Step 5. Whenever the mixture is even, spray the cold gas into the granulator till the gel is solidified, and obtain the pellet core wrapped with a hard capsule shell;

7) Weigh and take the pharmaceutical auxiliary ingredients and pellet core in line with the formula proportion, spray the syrup made in step 1) during the granulating process. Whenever the mixture is even, either pills or powder will be obtained in line with the size of pills.

8) Before the consolidation of syrup, deliver the finished products of Step 7 to the tablet press so as to obtain the final tablet.

In step 2), the starch, the individual mass ratio of sucrose and bacterial powder in the pellet core is subject to the tablet formula.

In step 7), the individual mass ratio of the pharmaceutical auxiliary ingredients, sucrose, starch and bacterial powder in the tablet is subject to the tablet formula.

1. The characteristics of the aforesaid live bacteria preparation of bacillus coagulans is its tablet form; this live bacterial tablet is made from the bacterial powder of bacillus coagulans HQ-1, which is coated with gelatin and cooled, and then made with other pharmaceutical auxiliary ingredients.

2. Aright-claim for the preparation method of such live bacteria preparation, characterized by the following steps:
1) Take sucrose and purified water for heating and dissolving, and then cool down to obtain the syrup, ready for follow-up use;
2) Under room temperature, put the starch in the granulation, and power on the granulator;
Meanwhile, spray syrup and the bacterial powder of bacillus coagulans HQ-1 until the required pellet cores are produced, and then take them out;
3) The core of the pellets shall be cooled under a temperature of 5-10° C. and be screened for follow-up use;
4) Take the medicine solid gelatin in the warm water bath and then cool it to room temperature to obtain the gel, ready for follow-up use;
5) Step 4) the gel shall be heated to obtain the solution;
6) Take the core of the pellet produced in the step 3, power on the pelletizer. Meanwhile, spray the solution made in the Step 5. Whenever the mixture is even, spray the cold gas into, the granulator till the gel is solidified, and obtain the pellet core wrapped with a hard capsule shell;
7) Weigh and take the pharmaceutical auxiliary ingredients and pellet core in line with the formula proportion, spray the syrup made in step 1) during the granulating process. Whenever the mixture is even, and then press into being tablets.

2. According to right-claim 1, the characteristics of the preparation method is the individual mass proportion of the starch, sucrose and bacterial powder inside the in the pellet core in step 2), subject to the tablet formula.

3. According to right-claim 1, the characteristics of the preparation method is the individual mass proportion of pharmaceutical excipients, sucrose, starch and bacterial powder in the tablet in in step 7), subject to the tablet formula.
